(19) United States
(12) Patent Application Publication
(10) Pub. No.: US 2003/0186843 A1
(43) Pub. Date: Oct. 2, 2003

(54) Powders for use in a dry powder inhaler

(76) Inventors: John Nicholas Staniforth, Bath (GB); David Alexander Vodden Morton, Bath (GB); Brian John Meakin, Bath (GB); David Ganderton, Exeter (GB)

Correspondence Address: DAVIDSON, DAVIDSON & KAPPEL, LLC, 485 SEVENTH AVENUE, 14TH FLOOR, NEW YORK, NY, 10018, US

(21) Appl. No.: 10/275,023

(22) PCT Filed: May 3, 2001
(86) PCT No.: PCT/GB01/01942

(30) Foreign Application Priority Data
May 3, 2000 (GB) 0010709.4

(51) Int. Cl. A61K 38/00; A61L 9/04; A61K 9/14; A61K 31/56; A61K 31/045

(52) U.S. Cl. 514/2; 424/46; 514/171; 514/729

(57) ABSTRACT

A powder for use in a dry powder inhaler comprises an active material and an indicator material that is capable of indicating to a patient that a dose of the active material has been administered. The powder for use in an inhaler device and/or an inhaler device containing the powder may be such that a fine particle fraction of at least 35% is generated.

This invention relates to powders for use in dry powder inhalers.

The use of inhaler devices for administering pharmaceutical products to the respiratory tract is well known. Inhalers are widely used particularly in the treatment of diseases of the respiratory tract. There are a number of types of inhaler currently available. In the pressurised metered dose inhaler, the dose to be administered is stored as a solution or a suspension in a liquid including low boiling point propellant (s). It is common for the patient, on administering such a dose to themselves, to experience a sensation of taste and/or cooling in the mouth and/or throat, due to the presence of the evaporating liquid propellants and/or co-solvents (additional liquids).

In the dry powder inhaler, the dose to be administered is stored in the form of a non-pressurised dry powder and, on actuation of the inhaler, the particles of the powder are inhaled by the patient. Dry powder inhaler devices include the Rotahaler and Diskhaler (Glaxo-Wellcome) and the Turbohaler (Astra-Draco).

In contrast to the pressurised metered dose inhaler, on administration of a dose to the patient from a dry powder inhaler it is usual for the patient to experience no sensation of taste, cooling or any other indication/confirmation that the dose has been successfully administered. Due to the generally fine particle size, the patient is often unaware of any powder impacting within the oropharyngeal region.

For the effective administration by a dry powder inhaler of the particles of active material to the lung where they can be absorbed, the particle size characteristics of the powder are particularly important.

In particular, for the effective delivery of active material deep into the lung, the active particles should be small and monodispersed on actuation of the inhaler.

The addition of materials as taste modifying agents is known in relation to compositions for use in types of inhaler device other than dry powder inhalers, for example in pressurised metered dose inhalers. The inclusion of taste-modifying agents has not been widely practised in the area of dry powder inhalers, however, because many of the materials used as taste modifiers are sticky solids or oils at room temperature. For example, Menthol BP has a melting point of 40 to 42° C. and tends to become sticky at body temperature. A thin film of the menthol would be “sticky” to the touch. The addition of such materials to a dry powder inhaler formulation would have been thought disadvantageous. In particular, agglomeration of the small active particles would have been expected, giving poorer performance of the powder and reduced clinical effect. Furthermore, the uncertainty as to the extent of agglomeration of the particles between each actuation of the inhaler, between different inhalers and different batches of powder, might have been expected to give poor dose reproductibility.

According to the invention there is released, on administration of the dose to a patient from a dry powder inhaler, additive material which indicates to the patient that the dose has been administered. The additive material, referred to below as indicator material, may be present in the powder as formulated for the dry powder inhaler, or be present in a separate form, such as in a separate location within the inhaler such that the additive becomes entrained in the airflow generated on inhalation simultaneously or sequentially with the powder containing the active material.

In a first aspect, the invention provides a powder for use in a dry powder inhaler, the powder comprising active material and further comprising an indicator material that is capable of indicating to a patient that a dose of the active material has been administered. The indicator material may form at least a partial coating on the surfaces of particles of the powder. The indicator material is advantageously a material which, when administered from the inhaler, induces a sensation within the oropharyngeal region.

The use of indicator material in accordance with the invention is of particular advantage where the active material is tasteless under the conditions of administration from the inhaler. It will be appreciated that references herein to the active material's being tasteless include references to active materials which are tasteless per se, and also include references to active materials which are capable of being tasted in some circumstances but are not tasted when administered from the inhaler, for example, because of the small size of the particles or because delivery of the active particles to the lungs is so effective that there is no residue in the oropharyngeal region to be tasted.

It has been found that it is possible in accordance with one embodiment of the invention to provide the sensation producing material as a coating on the surfaces of the powder particles.

As mentioned above, where the indicator material is an oil or sticky solid, it would have been expected that the performance of the powder would be unacceptably reduced. In fact, it has been found that, in some respects, the performance of the powder can be improved on addition of the indicator material. In particular, in some powder compositions, it has been found that the respirable fraction of the active material is increased on addition of indicator material in accordance with the present invention. The respirable fraction may be increased by a factor of more than 1.3.

The respirable fraction is a measure of the proportion of active material emitted from an inhaler device which would reach the deep lung in a patient. In examples of powders in accordance with the present invention including menthol as indicator material, the respirable fraction measured using the Glass Twin Impinger was increased to 40 to 45% compared with powders without the menthol for which the respirable fraction was less than 30%.

It is to be understood that the word “coating” used in relation to the indicator material on the surfaces of the particles is not to be interpreted to mean exclusively that the element forms a continuous envelope around the particle, indeed, in many cases, the indicator material would cover only limited portions of each “coated” particle thus forming a discontinuous covering. Some proportion of the indicator ingredient may also penetrate into the particle.

Where the indicator material forms a coating on the surfaces of, for example, particles of the active material, it is to be understood that further indicator material (which may be of the same or of a different composition) may be present in the powder as a coating on other particles of the powder and/or as discrete particles in the powder.

It is preferred for the powder to be such that a fine particle fraction of at least 35% is generated on actuation of the inhaler device. Thus, the invention also provides a powder for use in an inhaler device, the powder comprising active material and an indicator material that is capable of indicating to a patient that a dose of the active material has been administered, the powder being such that it generates a fine particle fraction of at least 35% on actuation of the inhaler device.

The term “fine particle fraction” is used herein to mean that fraction of the total amount of active material delivered by a device which has a diameter of not more than 5 μm. The total amount of active material delivered by a device is in general less than the amount of the active material that is metered in the device or is present in a pre-metered dose within the device.

Fine particle fractions referred to herein in relation to powders are as measured using a sample of the powder fired from a Cyclohaler into a Multi Stage Liquid Impinger (Apparatus C, European Pharmacopoeia, Method 5.2.9.18, Supplement 2000), although for convenience any other suitable measurement technique may be used.

The powder is preferably such that a fine particle fraction of at least 50% is generated on actuation of the inhaler device.

The invention also provides a dry powder inhaler, which contains active material and indicator material that is capable of indicating to a patient that a dose of the active material has been administered.

Moreover, the invention provides an inhaler device comprising a powder suitable for inhalation the powder comprising active material and an indicator material that is capable of indicating to a patient that a dose of the active material has been administered, and the inhaler device being a high turbulence inhaler device, the arrangement being such that a fine particle fraction of at least 35%, and preferably 50%, is generated on actuation of the inhaler device.

A “high turbulence inhaler device” is to be understood as meaning an inhaler device which is configured to generate relatively high turbulence within the device and/or a relatively high incidence of impaction of powder upon internal surfaces and/or obstructions within the device, whereby efficient de-agglomeration of agglomerated powder particles occurs in use of the device.

The invention offers particular advantages in the case of powders and/or inhalers which generate a relatively high fine particle fraction because it may not be immediately apparent to the user that a dose has been correctly administered. For example, the proportion of larger particles of active material may be too small to be effectively detected by taste or other sensory means.

Fine particle fractions generated from the powders and inhalers of the invention are advantageously up to 98%.

In one preferred form of inhaler, the active material is present in the form of a powder which additionally comprises the indicator material.

In another embodiment of the invention there is provided in the inhaler an indicator material which is not part of the powder. In that case the indicator material may be held within the inhaler such that it is able to provide a particulate or vapour release which is carried in the air stream passing through the inhaler. The indicator material may be located in the main air stream channel or a minor air stream channel of the inhaler. The indicator material may be held within a partially sealed region, which is sealed by valves that are only opened on inhalation through the device. The indicator material may be protected by any other suitable means.

The indicator material may comprise one or more substances.

The indicator material is advantageously a material which, when administered from the inhaler, induces a sensation within the oropharyngeal region, for example, a sensation of taste or temperature change or an olfactory sensation. Preferably, the sensation includes taste.

Indicator materials which are especially suitable for use in accordance with the invention include materials which are volatile or are oils or sticky solids at room temperature and/or body temperature, in particular terpenoids. Menthol and peppermint oil are particularly preferred indicator materials as well as other terpenes.

Also especially suitable are materials which have a relatively high negative heat of solution, and which as a result generate a cooling sensation in the patient's mouth on administration. For example, the material may be one having a negative heat of solution of at least 15 cal/g (62.5 J/g) and especially at least 20 cal/g (83.7 J/g), that is, a heat of solution of −15 cal/g (−62.5 J/g) or less and especially −20 cal/g (−83.7J/g) or less. Such materials include, for example, sorbitol (heat of solution −26 cal/g; −109 J/g), mannitol (−29 cal/g; −121.5 J/g), xylitol (−37 cal/g; −155 J/g), and erythritol (−43 cal/g; −180 J/g). Also suitable are materials that generate a cooling sensation by other means, for example, materials having a significant negative heat of evaporation, heat of sublimation or heat of melting, the cooling sensation then being a result of evaporation, sublimation or melting, respectively, in the oropharyngeal region.

The active material will comprise an effective amount of at least one active agent that has therapeutic activity when delivered into the lung. The active material advantageously consist essentially of one or more therapeutically active agents. Suitable therapeutically active agents may be drugs for therapeutic and/or prophylactic use. Active agents which may be included in the formulation include those products which are usually administered orally by inhalation for the treatment of disease such a respiratory disease, for example, β-agonists.

The active material may comprise at least one β₂-agonist, for example one or more compounds selected from terbutaline, salbutamol, salmeterol and formoterol. If desired, the active material may comprise more than one of those active agents, provided that they are compatible with one another under conditions of storage and use. Preferably, the active material are particles of salbutamol sulphate. References herein to any active agent are to be understood to include any physiologically acceptable derivative. In the case of the β₂-agonists mentioned above, physiologically acceptable derivatives include especially salts, including sulphates.

The active material may be particles of ipatropium bromide.

The active material may include a steroid, which may be beclometasone dipropionate or may be fluticasone. The active principle may include a cromone which may be sodium cromoglycate or nedocromil. The active principle may include a leukotriene receptor antagonist.

The active material may include a carbohydrate, for example heparin.

The active material may advantageously comprise a therapeutically active agent for systemic use provided that that agent is capable of being absorbed into the circulatory system via the lungs. For example, the active material may comprise peptides or polypeptides or proteins such as DNase, leukotrienes or insulin (including substituted insulins and pro-insulins), cyclosporin, interleukins, cytokines, anti-cytokines and cytokine receptors, vaccines (including influenza, measles, ‘anti-narcotic’ antibodies, meningitis), growth hormone, leuprolide and related analogues, interferons, desmopressin, immunoglobulins, erythropoeitin, calcitonin and parathyroid hormone. The formulation of the invention may in particular have application in the administration of insulin to diabetic patients, thus avoiding the normally invasive administration techniques used for that agent.

The formulations of the invention may advantageously be for use in pain relief. Non-opioid analgesic agents that may be included as pain relief agents are, for example, alprazolam, amitriptyline, aspirin, baclofen, benzodiazepines, bisphosphonates, caffeine, calcitonin, calcium-regulating agents, carbamazepine, clonidine, corticosteroids, dantrolene, dexamethasone, disodium pamidronate, ergotamine, flecainide, hydroxyzine, hyoscine, ibuprofen, ketamine, lignocaine, lorazepam, methotrimeprazine, methylprednisolone, mexiletine, mianserin, midazolam, NSAIDs, nimodipine, octreotide, paracetamol, phenothiazines, prednisolone, somatostatin. Suitable opioid analgesic agents are: alfentanil hydrochloride, alphaprodine hydrochloride, anileridine, bezitramide, buprenorphine hydrochloride, butorphanol tartrate, carfentanil citrate, ciramadol, codeine, dextromoramide, dextropropoxyphene, dezocine, diamorphine hydrochloride, dihydrocodeine, dipipanone hydrochloride, enadoline, eptazocine hydrobromide, ethoheptazine citrate, ethylmorphine hydrochloride, etorphine hydrochloride, fentanyl citrate, hydrocodone, hydromorphone hydrochloride, ketobemidone, levomethadone hydrochloride, levomethadyl acetate, levorphanol tartrate, meptazinol hydrochloride, methadone hydrochloride, morphine, nalbuphine hydrochloride, nicomorphine hydrochloride, opium, hydrochlorides of mixed opium alkaloids, papaveretum, oxycodone, oxymorphone hydrochloride, pentamorphone, pentazocine, pethidine hydrochloride, phenazocine hydrobromide, phenoperidine hydrochloride, picenadol hydrochloride, piritramide, propiram furmarate, remifentanil hydrochloride, spiradoline mesylate, sufentanil citrate, tilidate hydrochloride, tonazocine mesylate, tramadol hydrochloride, trefentanil.

The powders could also be used for the local administration of other agents for example for anti cancer activity, anti-virals, antibiotics, muscle relaxants, antidepressants, antiepileptics or the local delivery of vaccines to the respiratory tract.

The proportion of active material in the powder will depend upon the identity of the active material, and will typically be from 0.01 to 90% by weight of the total weight of the powder. In general the proportion of active material will not exceed 80% by weight of the total powder weight. For certain drugs, for example, β₂-agonists or corticosteroids, the proportion will typically be less than 10%, for example less than 5%, by weight based on the total powder weight.

While the proportion of other components in the powder will depend to some extent on the active material and the type of inhaler device to be used, the powder will advantageously include at least 0.1% by weight of indicator material based on the weight of the powder. Preferably the powder will include not more than 10%, for example, not more than 5%, by weight of the indicator material.

In many powder compositions for dry powder inhalers, the powder contains relatively large carrier particles and the small particles of active material adhere to the surfaces of the carrier particles before actuation of the inhaler but are dispersed from the surfaces on inhalation of the powder. Such powder compositions are widely used because the large size of the carrier particles helps to improve the flow properties of the powder.

Thus, in addition to the active material and the indicator material, the powder may further include carrier particles for carrying the particles of active material.

The carrier particles may be composed of any pharmacologically inert material or combination of materials which is acceptable for inhalation. Advantageously, the carrier particles are composed of one or more crystalline sugars; the carrier particles may be composed of one or more sugar alcohols or polyols. Preferably, the carrier particles are particles of dextrose or lactose, especially lactose.

Advantageously, at least 90% by weight of the carrier particles have a particle size which lies between 20 μm and 100 μm, more preferably between 60 μm and 100 μm. Preferably the size of at least 90% by weight of the carrier particles is less than 100 μm and lies between 60 μm and 1000 μm. The relatively large size of the carrier particles gives good flow and entrainment characteristics.

The powder may contain fine particles of an excipient material, which may for example be a material such as one of those mentioned above as being suitable or use as a carrier material, especially a crystalline sugar such as dextrose or lactose. The fine excipient material may be of the same or a different material from the carrier particles, where both are present. The particle size of the fine excipient material will generally not exceed 30 μm, and preferably does not exceed 20 μm.

In some circumstances, for example, where any carrier particles and/or any fine excipient material present is of a material itself capable of inducing a sensation in the oropharyngeal region, the carrier particles and/or the fine excipient material can constitute the indicator material. For example, carrier particles and/or any fine particle excipient may be of mannitol.

Where present, the amount of carrier particles will generally be up to 95%, for example, up to 90%, advantageously up to 80% and preferably up to 50% by weight based on the total weight of the powder. The amount of any fine excipient material, if present, may be up to 50% and advantageously up to 30%, especially up to 20%, by weight, based on the total weight of the powder.

Advantageously, at least 90% by weight of the active material has a particle size of not more than 10 μm, preferably not more than 5 μm. The particles therefore give a good suspension on actuation of the inhaler.

Where reference is made to particle size of particles of the powder, it is to be understood, unless indicated to the contrary, that the particle size is the aerodynamic particle size. The particle size may be calculated by a laser diffraction method.

Where the particle also includes an indicator material on the surface of the particle, advantageously the particle size of the coated particles is also within the preferred size ranges indicated for the uncoated particles.

Most advantageously, where the powder includes carrier particles, the indicator material forms at least a partial coating on the surfaces of carrier particles. Preferably at least 75%, more preferably at least 90% by weight of the indicator material is in the form of a coating on carrier particles, more preferably at least 95% by weight. Ideally, at least 90% and preferably substantially all of the indicator material is in the form of a coating on particles of the powder which have a size greater than 20 μm.

While it is clearly desirable for as large a proportion as possible of the particles of active material to be delivered to the deep lung, it is usually preferable for as little as possible of the other components to penetrate the deep lung. Indeed, for the indicator material to be sensed by the patient on actuation of the inhaler, the indicator material should normally be deposited in the mouth or throat. Where the indicator material is itself particulate, the particles are preferably of a size which is such that they will be deposited exclusively or substantially in the mouth and/or throat. That ensures that their presence will be perceived reliably by the patient and also reduces or eliminates the possibility of delivery of indicator material into the lung. Preferably therefore, the particles are of a particle size of at least 10 μm.

Thus, in a particularly preferred embodiment of the invention, the powder includes particles of active material, carrier particles for carrying the particles of active material and an indicator material, the indicator material forming at least a partial coating on the surfaces of carrier particles of the powder.

Where the powder material includes carrier particles, the proportions of the carrier particles, active particles and indicator material will depend to some extent on the active material and the inhaler device to be used.

In general, the powder material includes from about 0.01% to 95% by weight of active material and 0.1% to 10% by weight indicator material based on the weight of the powder. In some circumstances, however, the amount of indicator material may exceed 10%. By way of example where, as mentioned above, any carrier particle or fine excipient material present is of a material that is itself capable of inducing a sensation in the oropharyngeal region and constitutes the indicator material, it may be desirable for the quantity of that material to exceed 10% by weight based on the total weight of the powder.

The powder may further comprise other additives, suitable for use in formulations for dry powder inhalers. Where present, such additives will not normally exceed 10% by weight of the powder. Particles of those other additives may or may not have a coating of indicator material on their surfaces. Advantageously, at least one flow improver is present. Advantageously, there is present as an additive a ternary agent which is capable of enhancing the release of active material from carrier particles, thereby improving the fine particle fraction. Suitable ternary agents are for example the additive materials described in WO96/23485.

According to the invention there are also provided particles for use in a powder for a dry powder inhaler, the particles having at least a partial coating of indicator material on the surfaces of the particles.

Preferably, the particles are carrier particles as defined above.

Also provided by the present invention is the use, in a dry powder inhaler device for the purpose of indicating to a patient that a dose of an active material has been successfully administered, of a material that is capable of inducing a perceptible sensation in the oropharyngeal region.

The invention also provides a method for producing particles for use in a dry powder inhaler, the method including the steps of applying at least a partial coating of an indicator material to the surfaces of powder particles.

As indicated above, the indicator material may be applied to carrier particles where such particles are to be present in the powder composition.

Thus, the method for preparation of a powder for inhalation in accordance with the present invention may comprise the following steps

(a) forming a coating on the surfaces of carrier particles of indicator material, and

(b) mixing the resulting coated carrier particles with particles of active material.

Preferably, the indicator material is applied in the form of a solution. After application, the solvent would be dried off. Preferably, the indicator material is applied in a non-aqueous solvent.

In one preferred method, menthol is applied to the surfaces of carrier particles by the following method. Menthol is dissolved in diethyl ether (or methylene chloride) and the solution is applied to particles of lactose and the solvent is allowed to dry off.

In some circumstances, the powder for inhalation may be prepared by mixing the components of the powder together. For example, the powder may be prepared by mixing together particles of active material and menthol.

The dry powder inhaler devices in which the powder compositions of the present invention will commonly be used include “single dose” devices, for example the Rotahaler, the Spinhaler and the Diskhaler in which individual doses of the powder composition are introduced into the device in, for example, a capsule, or a blister and also multiple dose devices, for example the Turbohaler in which, on actuation of the inhaler, one dose of the powder is removed from a reservoir of the powder material contained in the device.

As already mentioned, in the case of certain powders, a form of device that promotes high turbulence offers advantages in that a higher fine particle fraction will be obtainable than in the use of other forms of device. Such devices include, for example, the Turbohaler (Trade Mark) or Novolizer (Trade Mark), and may be devices of the kind in which generation of an aerosolized cloud of powder is driven by inhalation of the patient or of the kind having a dispersal device for generating or assisting in generation of the aerosolized cloud of powder for inhalation.

“Actuation of the inhaler” refers to the process during which a dose of the powder is removed from its rest position in the inhaler, usually by a patient inhaling. That step takes place after the powder has been loaded into the inhaler ready for use.

The following Examples illustrate the invention.

Powders in accordance with the invention were prepared as follows: The percentages given (unless indicated to the contrary) are percentages by weight based on the total weight of the powder.

### EXAMPLE 1

| | |
|---|---|
| Sodium cromoglycate | 50.0% |
| Lactose | 48.5% |
| Menthol | 1.5% |

The menthol was dissolved in methylene chloride and the resulting solution was slowly added to the lactose particles while gently mixing. The resulting mixture was allowed to dry at room temperature. The coated lactose was mixed with the sodium cromoglycate by geometric dilution to form the powder. The formulation was aerosolised from a Turbohaler (Trade Mark) that being a device providing a high degree of de-agglomeration on actuation.

### EXAMPLE 2

Samples of lactose were produced in the size range 150 to 60 μm by sieving. 96.5 g of the sieved lactose was blended with 2 g of L-leucine in a Turbula mixer for 10 minutes. This mixture was transferred to a porcelain ball mill with 200 ml of 3 mm steel balls. The sample was milled at 60 rpm for 6 hours.

The sample was recovered from the mill. 10 g were treated with menthol as described in Example 1.

The menthol treated powder was blended by hand with the untreated lactose-leucine powder.

The resulting powder was hand blended with beclomethasone dipropionate to provide powder with the following composition:

| | |
|---|---|
| Beclomethasone dipropionate | 0.5% |
| Lactose | 96.5% |
| Menthol | 1.0% |
| Leucine | 2.0% |

### EXAMPLE 3

Samples of lactose were produced in the size range 150 to 60 μm by sieving. 94 g of the sieved lactose was blended with 2 g of L-leucine in a Turbula mixer for 10 minutes. This mixture was transferred to a porcelain ball mill with 200 ml of 3 mm steel balls. The sample was milled at 60 rpm for 6 hours.

The sample was recovered from the mill. 10 g were treated with menthol as described in Example 1.

The menthol treated powder was blended by hand with the untreated lactose-leucine powder.

The resulting powder was hand blended with salbutamol sulphate to provide powders with the following composition:

| | |
|---|---|
| Salbutarnol Sulphate | 2.0% |
| Lactose | 94.0% |
| Menthol | 2 .0% |
| Leucine | 2.0% |

### EXAMPLE 4

| | |
|---|---|
| Insulin | 49.0% |
| Lactose | 49.0% |
| Menthol | 2.0% |

The powder was prepared by the method of Example 1 above.

### EXAMPLE 5

| | |
|---|---|
| Sodium cromoglycate | 80.0% |
| Lactose | 11.5% |
| Saccharin sodium | 6.5% |
| Menthol | 2.0% |

The menthol was dissolved in methylene chloride. The lactose and saccharin sodium were mixed geometrically, the menthol solution was added and the mixture was allowed to dry at room temperature. The coated lactose/saccharin sodium was mixed with the sodium cromoglycate by geometric dilution. The formulation was aerosolised from a device providing a high degree of de-agglomeration on release.

### EXAMPLE 6

| | |
|---|---|
| Sodium cromaglycate | 25.0% |
| Lactose | 73.5% |
| Peppermint Oil | 1.5% |

The peppermint oil was dissolved in diethyl ether and the resulting solution was added to the lactose while gently mixing. The mixture was allowed to dry at room temperature. The coated lactose was mixed with the sodium cromoglycate by geometric dilution. The formulation was aerosolised from a device providing a high degree of de-agglomeration on release.

### EXAMPLE 7

Samples of lactose were produced in the size range 150 to 60 μm by sieving. 87.5 g of thus lactose was blended with 2 g of L-leucine in a Turbula mixer for 10 minutes. This mixture was transferred to a porcelain ball mill with 200 ml of 3 mm steel balls. The sample was milled at 60 rpm for 6 hours.

The sample was recovered from the mill log were treated with menthol as described in Example 1.

The menthol treated powder was blended by hand with the untreated lactose-leucine powder.

The resulting powder was hand blended with flunisolide to provide powders with the following composition:

| | |
|---|---|
| Flunisolide | 10.0% |
| Lactose | 87.5% |
| Menthol | 0.5% |
| Leucine | 2.0% |

1. A powder for use in a dry powder inhaler, the powder comprising active material and further comprising indicator material that is capable of indicating to a patient that a dose of the active material has been administered.

2. A powder according to claim 1, wherein the indicator ingredient forms at least a partial coating on the surfaces of the particles of the powder.

3. A powder according to claim 1, wherein the indicator material is a material which, when administered from the inhaler, induces a sensation within the oropharyngeal region.

4. A powder according to claim 3, wherein the sensation induced is of taste.

5. A powder according to claim 3 or claim 4, wherein a sensation of temperature change is induced.

6. A powder according to any one of claims 3 to 5, wherein an olfactory sensation is induced.

7. A powder according to any one of claims 1 to 6, wherein the active the active material is substantially tasteless.

8. A powder according to any one of claims 1 to 7, wherein the active material comprises a steroidal drug.

9. A powder according to any one of claims 1 to 8, wherein the active material comprises a β₂-agonist.

10. A powder according to any one of claims 1 to 9, wherein the active material comprises an antimuscarinic drug.

11. A powder according to any one of claims 1 to 10, wherein the active material comprises a peptide or polypeptide.

12. A powder according to any one of claims 1 to 11, wherein the indicator material comprises menthol.

13. A powder according to any one of claims 1 to 12, wherein the powder includes at least 0.1% by weight indicator material based on the weight of the powder.

14. A powder according to any one of claims 1 to 13, wherein the powder includes up to 10% by weight indicator material, based on the total weight of the powder.

15. A powder according to any preceding claim, wherein the powder further includes carrier particles for carrying the particles of active material.

16. A powder according to claim 13, wherein the carrier particles comprise one or more crystalline sugars.

17. A powder according to claim 15 or claim 16, wherein at least 90% by weight of the carrier particles have a size between 20 μm and 1000 μm.

18. A powder according to any of claims 15 to 17, wherein at least 90% by weight of the indicator material is in the form of a coating on the surfaces of particles having a size greater than 20 μm.

19. A powder according to any one of claims 1 to 19, wherein the indicator material comprises a terpenoid.

20. A powder according to claim 20, in which the indicator material comprises menthol or peppermint oil.

21. A powder according to any one of claims 1 to 20, which additionally comprises fine particles of an excipient material.

22. A powder according to any one of claims 1 to 21, in which the indicator material also acts as carrier particles and/or as a fine excipient material.

23. A powder according to any one of claims 1 to 22, which further comprises an additive material for promoting the release of active particles from the powder on actuation of the inhaler.

24. A powder for use in a dry powder inhaler, the powder including particles of active material, carrier particles for carrying the particles of active material and an indicator, the indicator material forming at least a partial coating on the surfaces of carrier particles in the powder.

25. A powder for use in an inhaler device, the powder comprising active material and an indicator material that is capable of indicating to a patient that a dose of the active material has been administered, the powder being such that it generates a fine particle fraction of at least 35%, preferably at least 50%, on actuation of the inhaler device.

26. A powder for use in a dry powder inhaler, the powder being substantially as described herein in any one of Examples 1 to 7.

27. Particles for use in a powder for a dry powder inhaler, the particles having at least a partial coating of an indicator material on the surfaces of the particles.

28. Particles according to claim 27, wherein the particles are carrier particles.

29. Particles for use in a powder for a dry powder inhaler, the particles being substantially as described herein in any one of Examples 1 to 7.

30. A dry powder inhaler, which contains active material and indicator material that is capable of indicating to a patient that a dose of the active material has been administered.

31. A dry powder inhaler according to claim 30, wherein the active material is present in the form of a powder according to any one of claims 1 to 24 or of particles according to any of claims 26 to 28.

32. A dry powder inhaler according to claim 30, wherein the indicator material is stored in the inhaler separately from the active powder.

33. A dry powder inhaler according to claim 32, wherein the indicator material is so arranged that it can be introduced into an air stream channel of the inhaler.

34. A dry powder inhaler according to claim 32 or claim 32, wherein the additive is held within a region that is at least partially sealed other than during inhalation.

35. A dry powder inhaler according to claim 34, wherein said region is sealed by valves that are openable on inhalation through the device.

36. An inhaler device comprising a powder for use in an inhaler device, the powder comprising active material and an indicator to a patient that a dose of the active material has been administered, and the inhaler device being a high turbulence inhaler device, the arrangement being such that a fine particle fraction of at least 35% is generated on actuation of the inhaler device.

37. A powder according to claim 36, in which the arrangement is such that a fine particle fraction of at least 50% is generated on actuation of the inhaler device.

38. Use, in a dry powder inhaler device for the purpose of indicating to a patient that a dose of an active material has been successfully administered, of a material that is capable of inducing a perceptible sensation in the oropharyngeal region.

39. A method of producing particles for use in a dry powder inhaler, the method including the step of applying at least a partial coating of an indicator material to the surfaces of particles of the powder.

40. A method according to claim 39, wherein the indicator material is applied to the surfaces of carrier particles.

41. A method according to claim 39 or claim 40, wherein the indicator material is applied in the form of a solution.

42. A method according to claim 41, wherein the indicator material is applied in solution with a non-aqueous solvent.

43. A method of producing a powder according to any of claims 1 to 26, the method being according to any of claims 39 to 42.

44. A method of producing particles according to any one of claims 27 to 29, the method being according to any of claims 39 to 42.

45. A method for producing a powder for use in a dry powder inhaler, the method being substantially as herein described.
